(19) United States
(12) Patent Application Publication
(10) Pub. No.: US 2015/0197807 A1
(43) Pub. Date: Jul. 16, 2015

(54) USE OF NANOWIRES FOR DELIVERING BIOLOGICAL EFFECTORS INTO IMMUNE CELLS

(71) Applicant: President and Fellows of Harvard College, Cambridge, MA (US)

(72) Inventors: Hongkun Park, Lexington, MA (US); Alexander K. Shalek, Cambridge, MA (US); Jellert T. Gaublomme, Cambridge, MA (US)

(73) Assignee: Presidents and Fellows of Harvard College, Cambridge, MA (US)

(21) Appl. No.: 14/422,497

(22) PCT Filed: Mar. 15, 2013
(86) PCT No.: PCT/US2013/032457

§ 371 Date: Feb. 19, 2015

Related U.S. Application Data
(60) Provisional application No. 61/684,918, filed on Aug. 20, 2012.

(51) Int. Cl. C12Q 1/68; G01N 33/50

(52) U.S. Cl. CPC C12Q 1/6883; G01N 33/502; C12Q 2600/158; G01N 2500/04; G01N 2500/10; G01N 2333/52

(57) ABSTRACT

The present invention generally relates to nanowires and, in some aspects, to methods of using nanowire arrays to identify a therapeutic target for treating a disorder in a subject, identify a treatment for a disorder in a subject, or deliver a biological effector to immune cells. Previous techniques for delivering biological effectors into live immune cells yielded low efficiencies, activated the immune response and induced non-specific inflammation, and/or required harsh conditions that resulted in widespread apoptosis. By contrast, some of the methods described herein are capable of efficiently delivering biomolecular cargo into immune cells, have negligible toxicity, do not activate immune cell function, and/or allow cells to respond appropriately to physiological stimuli.

### RELATED APPLICATIONS

This application claims the benefit of U.S. Provisional Patent Application Ser. No. 61/684,918, filed Aug. 20, 2012, entitled “Use of Nanowires for Delivering Biological Effectors into Immune Cells,” by Hongkun Park, et al., incorporated herein by reference.

### GOVERNMENT FUNDING

Research leading to various aspects of the present invention was sponsored, at least in part, by the National Institutes of Health, Contract Nos. 1P50HG006193-01 and 8DP1DA035083-05. The U.S. Government has certain rights in the invention.

### FIELD

The present invention generally relates to nanowires and, in some aspects, to methods of using nanowire arrays to identify a therapeutic target for treating a disorder in a subject, identify a treatment for a disorder in a subject, or deliver a biological effector to immune cells.

### BACKGROUND

Achieving a circuit-level understanding of cellular function requires techniques to systematically perturb intracellular components and measure cellular responses. Since many perturbing agents, such as plasmid deoxyribonucleic acids (DNAs), small interfering ribonucleic acids (siRNAs), peptides, and proteins, do not spontaneously cross the cell membrane with high efficiency, one of the challenges has been developing methods to deliver these biological effectors into living cells. This has been a particular challenge in primary immune cells, in part due to their propensity to activate or undergo apoptosis (cell death) in the presence of foreign substances or when sensing damage or danger signals. Moreover, different immune cells (e.g., macrophages and T cells), in addition to performing unique functions through a variety of distinct functional mechanisms, can possess vastly different morphologies, sizes, and adhesive properties. Current methods of delivery into immune cells have yielded low efficiencies, activated the immune response and induced non-specific inflammation, and/or required harsh conditions that resulted in widespread apoptosis. This resistance to conventional transfection has been a major stumbling block in using perturbations, such as RNA interference (RNAi), to characterize primary immune cell and tumor function, despite the availability of both human and murine samples in healthy and disease states. There is a need to develop efficient, minimally invasive approaches for delivering biological effectors to immune cells.

### SUMMARY

The present invention generally relates to nanowires and, in some aspects, to methods of using nanowire arrays to identify a therapeutic target for treating a disorder in a subject, identify a treatment for a disorder in a subject, or deliver a biological effector to immune cells. The subject matter of the present invention involves, in some cases, interrelated products, alternative solutions to a particular problem, and/or a plurality of different uses of one or more systems and/or articles.

In one aspect, the invention is related to a method of identifying a therapeutic target for treating a disorder, comprising: providing upstanding nanowires (NWs) in an array; coating the NWs with a biological effector for modulating expression or activity of a cellular target; contacting immune cells atop the array so that at least some of the immune cells are penetrated by one or more NWs, the immune cells being related to the disorder; incubating the immune cells for a period of time to allow for release of the biological effector into the penetrated immune cells; assessing a phenotype of the immune cells; and determining whether the cellular target is a therapeutic target for treating the disorder based on the phenotype, wherein the average lengths, average diameters, and density of the nanowires are configured to permit adhesion and subsequent penetration of the immune cells. In some embodiments, at least some of the nanowires are silicon nanowires. In certain embodiments, the biological effector is a small molecule, a DNA molecule, an RNA molecule, or a protein. In some embodiments, the average length of the nanowires is 0.1-10 micrometers (μm), and/or the diameter of the nanowires is 50-300 nm, and/or the density of the nanowires is 0.05-5 nanowires per micrometer (μm²).

In another aspect, the invention is related to a method of identifying a treatment for a disorder in a subject, comprising: providing upstanding NWs in an array; coating the NWs with a compound for treating the disorder; contacting immune cells obtained from the subject atop the array so that at least some of the immune cells are penetrated by one or more NWs, the immune cells being related to the disorder; incubating the immune cells for a period of time to allow for release of the compound into the penetrated immune cells; assessing a phenotype of the immune cells; and determining whether the compound would be effective for treating the disorder in the subject based on the phenotype, wherein the average lengths, average diameters, and density of the nanowires are configured to permit adhesion and subsequent penetration of the immune cells.

In another aspect, the invention is related to a method of delivering a biological effector to immune cells, comprising: providing upstanding NWs in an array; coating the NWs with a biological effector; contacting immune cells atop the array so that at least some of the immune cells are penetrated by one or more NWs; and incubating the cells for a period of time to allow for release of the biological effector into the penetrated cells, wherein the average lengths, average diameters, and density of the nanowires are configured to permit adhesion and subsequent penetration of the immune cells.

In another set of embodiments, the present invention is generally directed to a method of silencing a gene in an immune cell. In certain embodiments, the method comprises providing upstanding nanowires in an array, at least some of the nanowires comprising siRNA coated thereon, inserting at least one of the upstanding nanowires into an immune cell, and incubating the immune cell for a time at least sufficient to activate the siRNA to silence the gene.

The present invention, in another set of embodiments, is directed to a method comprising providing a plurality of substrates, each of which comprises upstanding nanowires in an array, at least some of which substrates comprise different biological effectors; depositing a plurality of cells on the plurality of substrates to insert the biological effectors into the plurality of cells; and determining phenotypes of the plurality of cells after insertion of the biological effectors.

In yet another set of embodiments, the present invention is generally directed to a method comprising inserting a plurality of upstanding nanowires on a substrate into a plurality of immune cells, at least some of the nanowires being at least partially coated with a biological effector, causing release of the biological effector internally of at least some of the immune cells, and determining a phenotype of at least some of the immune cells.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 provides images of nanowires penetrating the cell membrane and delivering siRNA into a variety of ex vivo primary immune cells.

FIG. 2 provides confocal scans showing delivery of a broad range of exogenous, labeled molecules, including DNA, peptides, proteins, and siRNA, into bone marrow-derived dendritic cells (BMDCs).

FIG. 3 provides confocal scans showing delivery of exogenous, labeled molecules, including DNA, peptides, proteins, and siRNA, into primary murine splenocytes.

FIG. 4 provides plots demonstrating that NW-mediated delivery is minimally invasive, yet effective, in ex vivo primary immune cells.

FIG. 5 provides images and a plot demonstrating that optimized NWs do not adversely affect BMDC viability.

FIG. 6 provides plots demonstrating that NWs and their cargo neither activate innate immune sensing nor inhibit normal responses to lipopolysaccharide (LPS) stimulation.

FIG. 7 provides plots demonstrating that oligonucleotides, plasmid DNA, small molecules, peptides, and proteins do not activate or inhibit innate immune responses in BMDCs.

FIG. 8 provides a plot demonstrating that human B cells will grow and divide on NWs when stimulated with IL-4 and CD40L.

FIG. 9 provides gene expression profiles that show global dysregulation of the Wnt pathway in chronic lymphocytic leukemia (CLL) samples compared to normal samples.

FIG. 10 provides images and plots demonstrating that NWs successfully delivered LEF1 siRNA into ex vivo human B cells obtained from normal donors and CLL patients, revealing functional heterogeneity that correlates with clinical outcome.

FIG. 11 provides a plot showing that CLL sample LEF1 expression does not correlate with magnitude of effect on sample viability following LEF1 knockdown.

FIG. 12 provides gene expression profiles showing that functional sample groupings are not uncovered by correlations based on clustering across Wnt pathway members.

FIG. 13 provides an expression map for 823 genes that are significantly different between high-, low-, and inverse-responders.

FIG. 14 shows microarray expression differences for selected genes that were tested using quantitative real-time polymerase chain reaction (qRT-PCR).

FIG. 15 shows clustering of all B cell samples for which microarray data was available.

FIG. 16 demonstrates that the extended response groups exhibited similar behaviors as the initial response groups, with no enrichment for any known CLL cytogenetic markers, but significantly different times to first therapy.

FIG. 17 shows single sample gene set enrichment analysis of three gene modules associated with hematopoietic stem cells and embryonic stem cells.

FIG. 18 shows a schematic for a potential mechanism for the observed effects of LEF1 knockdown.

### DETAILED DESCRIPTION

The present invention generally relates to nanowires and, in some aspects, to methods of using nanowire arrays to identify a therapeutic target for treating a disorder in a subject, identify a treatment for a disorder in a subject, or deliver a biological effector to immune cells. Previous techniques for delivering biological effectors into live immune cells yielded low efficiencies, activated the immune response and induced non-specific inflammation, and/or required harsh conditions that resulted in widespread apoptosis. By contrast, some of the methods described herein are capable of efficiently delivering biomolecular cargo into immune cells, have negligible toxicity, do not activate immune cell function, and/or allow cells to respond appropriately to physiological stimuli.

In some aspects, the methods are capable of modulating expression or activity of a cellular target or set of targets and assessing the phenotypic consequences, allowing identification of cellular targets that hold promise as therapeutic targets in different diseases. Importantly, in certain cases, the efficacy and universality of the delivery technique enables the discovery process to be performed in the actual diseased cells, even if they are difficult-to-transfect primary immune cells, rather than in cell lines. Since many times a single disease manifests from multiple origins, the utility of targeting a specific target in any given patient can be tested before beginning treatment.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Various aspects of this invention involve an array of upstanding nanowires. On average, the upstanding nanowires may form an angle with respect to a substrate of between about 80° and about 100°, between about 85° and about 95°, or between about 88° and about 92°. In some cases, the average angle is about 90°. As used herein, the term “nanowire” (or “NW”) refers to a material in the shape of a wire or rod having a diameter in the range of 1 nm to 1 micrometer (μm). The NWs may be formed from materials with low cytotoxicity; suitable materials include, but are not limited to, silicon, silicon oxide, silicon nitride, silicon carbide, iron oxide, aluminum oxide, iridium oxide, tungsten, stainless steel, silver, platinum, and gold. Other suitable materials include aluminum, copper, molybdenum, tantalum, titanium, nickel, tungsten, chromium, or palladium. In some embodiments, the nanowire comprises or consists essentially of a semiconductor. Typically, a semiconductor is an element having semiconductive or semi-metallic properties (i.e., between metallic and non-metallic properties). An example of a semiconductor is silicon. Other non-limiting examples include elemental semiconductors, such as gallium, germanium, diamond (carbon), tin, selenium, tellurium, boron, or phosphorous. In other embodiments, more than one element may be present in the nanowires as the semiconductor, for example, gallium arsenide, gallium nitride, indium phosphide, cadmium selenide, etc.

The size and density of the NWs in the NW arrays may be varied; the lengths, diameters, and density of the NWs can be configured to permit adhesion and penetration of immune cells. In some embodiments, the length of the NWs can be 0.1-10 micrometers (μm). In some cases, the diameter of the NWs can be 50-300 nm. In certain embodiments, the density of the NWs can be 0.05-5 NWs per micrometer (μm²). Other examples are discussed below.

The nanowires may be regularly or irregularly spaced on the substrate. For example, the nanowires may be positioned within a rectangular grid with periodic spacing, e.g., having a periodic spacing of at least about 0.01 micrometers, at least about 0.03 micrometers, at least about 0.05 micrometers, at least about 0.1 micrometers, at least about 0.3 micrometers, at least about 0.5 micrometers, at least about 1 micrometer, at least about 2 micrometers, at least about 3 micrometers, at least about 5 micrometers, at least about 10 micrometers, etc. In some cases, the periodic spacing may be no more than about 10 micrometers, no more than about 5 micrometers, no more than about 3 micrometers, no more than about 1 micrometer, no more than about 0.5 micrometers, no more than about 0.3 micrometers, no more than about 0.1 micrometers, no more than about 0.05 micrometers, no more than about 0.03 micrometers, no more than about 0.01 micrometers, etc. Combinations of these are also possible, e.g., the array may have a periodic spacing of nanowires of between about 0.01 micrometers and about 0.03 micrometers.

In some cases, the nanowires (whether regularly or irregularly spaced) may be positioned on the substrate such that the average distance between a nanowire and its nearest neighboring nanowire is at least about 0.01 micrometers, at least about 0.03 micrometers, at least about 0.05 micrometers, at least about 0.1 micrometers, at least about 0.3 micrometers, at least about 0.5 micrometers, at least about 1 micrometer, at least about 2 micrometers, at least about 3 micrometers, at least about 5 micrometers, at least about 10 micrometers, etc. In some cases, the distance may be no more than about 10 micrometers, no more than about 5 micrometers, no more than about 3 micrometers, no more than about 1 micrometer, no more than about 0.5 micrometers, no more than about 0.3 micrometers, no more than about 0.1 micrometers, no more than about 0.05 micrometers, no more than about 0.03 micrometers, no more than about 0.01 micrometers, etc. In some cases, the average distance may fall within any of these values, e.g., between about 0.5 micrometers and about 2 micrometers.

The nanowires may have any suitable length, as measured moving away from the substrate. The nanowires may have substantially the same lengths, or different lengths in some cases. For example, the nanowires may have an average length of at least about 0.1 micrometers, at least about 0.2 micrometers, at least about 0.3 micrometers, at least about 0.5 micrometers, at least about 0.7 micrometers, at least about 1 micrometer, at least about 2 micrometers, at least about 3 micrometers, at least about 5 micrometers, at least about 7 micrometers, or at least about 10 micrometers. In some cases, the nanowires may have an average length of no more than about 10 micrometers, no more than about 7 micrometers, no more than about 5 micrometers, no more than about 3 micrometers, no more than about 2 micrometers, no more than about 1 micrometer, no more than about 0.7 micrometers, no more than about 0.5 micrometers, no more than about 0.3 micrometers, no more than about 0.2 micrometers, or no more than about 0.1 micrometers. Combinations of any of these are also possible in some embodiments.

The nanowires may also have any suitable diameter, or narrowest dimension if the nanowires are not circular. The nanowires may have substantially the same diameters, or in some cases, the nanowires may have different diameters. In some cases, the nanowires may have an average diameter of at least about 10 nm, at least about 30 nm, at least about 50 nm, at least about 70 nm, at least about 100 nm, at least about 200 nm, at least about 300 nm, etc., and/or the nanowires may have an average diameter of no more than about 300 nm, no more than about 200 nm, no more than about 100 nm, no more than about 70 nm, no more than about 50 nm, no more than about 30 nm, no more than about 20 nm, or no more than about 10 nm, or any combination of these.

In addition, in some cases, the density of nanowires on the substrate, or on a region of the substrate defined by nanowires, may be at least about 0.01 nanowires per square micrometer, at least about 0.02 nanowires per square micrometer, at least about 0.03 nanowires per square micrometer, at least about 0.05 nanowires per square micrometer, at least about 0.07 nanowires per square micrometer, at least about 0.1 nanowires per square micrometer, at least about 0.2 nanowires per square micrometer, at least about 0.3 nanowires per square micrometer, at least about 0.5 nanowires per square micrometer, at least about 0.7 nanowires per square micrometer, at least about 1 nanowire per square micrometer, at least about 2 nanowires per square micrometer, at least about 3 nanowires per square micrometer, at least about 4 nanowires per square micrometer, at least about 5 nanowires per square micrometer, etc. In addition, in some embodiments, the density of nanowires on the substrate may be no more than about 10 nanowires per square micrometer, no more than about 5 nanowires per square micrometer, no more than about 4 nanowires per square micrometer, no more than about 3 nanowires per square micrometer, no more than about 2 nanowires per square micrometer, no more than about 1 nanowire per square micrometer, no more than about 0.7 nanowires per square micrometer, no more than about 0.5 nanowires per square micrometer, no more than about 0.3 nanowires per square micrometer, no more than about 0.2 nanowires per square micrometer, no more than about 0.1 nanowires per square micrometer, no more than about 0.07 nanowires per square micrometer, no more than about 0.05 nanowires per square micrometer, no more than about 0.03 nanowires per square micrometer, no more than about 0.02 nanowires per square micrometer, or no more than about 0.01 nanowires per square micrometer.

The substrate may be formed of the same or different materials as the nanowires. For example, the substrate may comprise silicon, silicon oxide, silicon nitride, silicon carbide, iron oxide, aluminum oxide, iridium oxide, tungsten, stainless steel, silver, platinum, gold, gallium, germanium, or any other materials described herein that a nanowire may be formed from. In one embodiment, the substrate is formed from a semiconductor.

In some embodiments, arrays of NWs on a substrate may be obtained by growing NWs from a precursor material. As a non-limiting example, chemical vapor deposition (CVD) may be used to grow NWs by placing or patterning catalyst or seed particles (typically with a diameter of 1 nm to a few hundred nm) atop a substrate and adding a precursor to the catalyst or seed particles. When the particles become saturated with the precursor, NWs can begin to grow in a shape that minimizes the system's energy. By varying the precursor, substrate, catalyst/seed particles (e.g., size, density, and deposition method on the substrate), and growth conditions, NWs can be made in a variety of materials, sizes, and shapes, at sites of choice.

In certain embodiments, arrays of NWs on a substrate may be obtained by growing NWs using a top-down process that involves removing predefined structures from a supporting substrate. As a non-limiting example, the sites where NWs are to be formed may be patterned into a soft mask and subsequently etched to develop the patterned sites into three-dimensional nanowires. Methods for patterning the soft mask include, but are not limited to, photolithography and electron beam lithography. The etching step may be either wet or dry.

In some embodiments of the invention, at least some of the NWs may undergo surface modification so that molecules of interest can be attached to them, e.g., for delivery into a cell. It should be appreciated that the NWs can be complexed with various molecules according to any method known in the art. It should also be appreciated that the molecules connected to different NWs may be distinct. In some embodiments, a NW may be attached to a molecule of interest through a linker. The interaction between the linker and the NW may be covalent, electrostatic, photosensitive, or hydrolysable. As a specific non-limiting example, a silane compound may be applied to a NW with a surface layer of silicon oxide, resulting in a covalent Si—O bond. As another specific non-limiting example, a thiol compound may be applied to a NW with a surface layer of gold, resulting in a covalent Au—S bond. Examples of compounds for surface modification include, but are not limited to, aminosilanes such as (3-aminopropyl)-trimethoxysilane, (3-aminopropyl)-triethoxysilane, 3-(2-aminoethylamino)propyl-dimethoxymethylsilane, (3-aminopropyl)-diethoxy-methylsilane, [3-(2-aminoethylamino)propyl]trimethoxysilane, bis[3-(trimethoxysilyl)propyl]amine, and (11-aminoundecyl)-triethoxysilane; glycidoxysilanes such as 3-glycidoxypropyldimethylethoxysilane and 3-glycidyloxypropyl)trimethoxysilane; mercaptosilanes such as (3-mercaptopropyl)-trimethoxysilane and (11-mercaptoundecyl)-trimethoxysilane; and other silanes such as trimethoxy(octyl)silane, trichloro(propyl)silane, trimethoxyphenylsilane, trimethoxy(2-phenylethyl)silane, allyltriethoxysilane, allyltrimethoxysilane, 3-[bis(2-hydroxyethyl)amino]propyl-triethoxydilane, 3-(trichlorosilyl)propyl methacrylate, and (3-bromopropyl)trimethoxysilane. Other non-limiting examples of compounds that may be used to form the linker include poly-lysine, collagen, fibronectin, and laminin.

In addition, in various embodiments, a nanowire may be prepared for binding or coating of a suitable biological effector by activating the surface of the nanowire, silanizing at least a portion of the nanowire, and reacting a crosslinker to the silanized portions of the nanowire. Methods for activating the surface include, but are not limited to, surface oxidation, such as by plasma oxidation or acid oxidation. Non-limiting examples of suitable types of crosslinkers that are commercially available and known in the art include maleimides, histidines, haloacetyls, and pyridyldithiols.

The interaction between the linker and the molecule to be delivered can be covalent, electrostatic, photosensitive, or hydrolysable. In some embodiments, a molecule of interest attached to or coated on a NW may be a biological effector. As used herein, a “biological effector” refers to a substance that is able to modulate the expression or activity of a cellular target. It includes, but is not limited to, a small molecule, a protein (e.g., a natural protein or a fusion protein), an enzyme, an antibody (e.g., a monoclonal antibody), a nucleic acid (e.g., DNA, including linear and plasmid DNAs; RNA, including mRNA, siRNA, and microRNA), and a carbohydrate. The term “small molecule” refers to any molecule with a molecular weight below 1000 Da. Non-limiting examples of molecules that may be considered to be small molecules include synthetic compounds, drug molecules, oligosaccharides, oligonucleotides, and peptides. The term “cellular target” refers to any component of a cell. Non-limiting examples of cellular targets include DNA, RNA, a protein, an organelle, a lipid, or the cytoskeleton of a cell. Other examples include the lysosome, mitochondria, ribosome, nucleus, or the cell membrane.

As mentioned, in one embodiment, the biological effector is siRNA. siRNA, or “Small Interfering RNA,” in general is a class of double-stranded RNA molecules, typically 20-25 base pairs in length. siRNA plays a role in the RNA interference (RNAi) pathway, where it interferes with the expression of specific genes with complementary nucleotide sequence. Thus, for example, siRNA may have a sequence that is antisense to a sequence within a target gene. siRNA also acts in RNAi-related pathways in some cases, e.g., as an antiviral mechanism or in shaping the chromatin structure of a genome. The siRNAs typically have a structure comprising a short (usually 21-bp) double-stranded RNA (dsRNA) with phosphorylated 5′ ends and hydroxylated 3′ ends with two overhanging nucleotides. siRNAs are typically produced by the Dicer enzyme reacting with various precursor RNAs. Those of ordinary skill in the art will be able to identify siRNAs, many of which have been cataloged in publically accessible databases.

In some cases, the nanowires can be used to deliver biological effectors or other suitable biomolecular cargo into a population of cells at surprisingly high efficiencies. Furthermore, such efficiencies may be achieved regardless of cell type, as the primary mode of interaction between the nanowires and the cells is physical insertion, rather than biochemical interactions (e.g., as would appear in traditional pathways such as phagocytosis, receptor-mediated endocytosis, etc.). For instance, in a population of cells on the surface of the substrate, at least about 50%, at least about 60%, at least about 70%, at least about 80%, or at least about 90% of the cells may have at least one nanowire inserted therein. In some cases, as discussed herein, the nanowires may have at least partially coated thereon one or more biological effectors. Thus, in some embodiments, biological effectors may be delivered to at least about 50%, at least about 60%, at least about 70%, at least about 80%, or at least about 90% of the cells on the substrate, e.g., via the nanowires.

In one set of embodiments, the surface of the substrate may be treated in any fashion that allows binding of cells to occur thereto. For example, the surface may be ionized and/or coated with any of a wide variety of hydrophilic and/or cytophilic materials, for example, materials having exposed carboxylic acid, alcohol, and/or amino groups. In another set of embodiments, the surface of the substrate may be reacted in such a manner as to produce carboxylic acid, alcohol, and/or amino groups on the surface. In some cases, the surface of the substrate may be coated with a biological material that promotes adhesion or binding of cells, for example, materials such as fibronectin, laminin, vitronectin, albumin, collagen, or peptides or proteins containing RGD sequences.

It should be understood that for a cell to adhere to the nanowire, a separate chemical or “glue” is not necessarily required. In some cases, sufficient nanowires may be inserted into a cell such that the cell cannot easily be removed from the nanowires (e.g., through random or ambient vibrations), and thus, the nanowires are able to remain inserted into the cells. In some cases, the cells cannot be readily removed via application of an external fluid after the nanowires have been inserted into the cells.

In some cases, merely placing or plating the cells on the nanowires is sufficient to cause at least some of the nanowires to be inserted into the cells. For example, a population of cells suspended in media may be added to the surface of the substrate containing the nanowires, and as the cells settle from being suspended in the media to the surface of the substrate, at least some of the cells may encounter nanowires, which may (at least in some cases) become inserted into the cells.

In certain embodiments, a molecule of interest may be delivered to a cell using a nanowire. The molecule of interest attached to or coated on a NW may be a compound for treating a disorder. As used herein, a “disorder” refers to an immune-regulated disorder or condition. Examples of disorders include, but are not limited to, an autoimmune disorder, an immunodeficiency, allergy, cancer, infection, or transplant rejection. Non-limiting examples of immune disorders include humoral immune deficiency, T cell deficiency, neutropenia, asplenia, or complement deficiency. Non-limiting examples of autoimmune disorders include lupus, scleroderma, hemolytic anemia, vasculitis, type I diabetes, Grave's disease, rheumatoid arthritis, multiple sclerosis, Goodpasture's syndrome, pernicious anemia, myopathy, etc.

As a non-limiting example of how the immune cells may be positioned, the immune cells may be plated on the NW array substrate using plating methods known in the art. After the molecules to be delivered have been attached to the NWs, in some aspects of the invention, immune cells are placed atop the NW array so that at least some of the immune cells are penetrated by one or more NWs. The nanowires may penetrate partially or completely into the cells, depending on factors such as the size or dimensions of the nanowire, the size or shape of the cells, etc. For example, the nanowires may be inserted into the cytosol of a cell, or into an organelle within the cell, such as into a mitochondria, a lysosome, the nucleus, or a vacuole.

In some embodiments, the delivery of molecules of interest into cells such as immune cells may be achieved through methods that include, but are not limited to, microarraying, stamping, applying masks, ink-jet printing, hand-printing, or controlling cell plating sites.

As used herein, “immune cells” refer to cells of the immune system, which defend the body against disease and foreign materials. Non-limiting examples of immune cells include dendritic cells, such as bone marrow-derived dendritic cells; lymphocytes, such as B cells, T cells, and natural killer cells; and macrophages. The immune cells may, in some embodiments, be derived from bone marrow, spleen, or blood from a suitable subject. For example, the immune cells may arise from a human or a non-human mammal, such as a monkey, ape, cow, sheep, goat, horse, donkey, llama, rabbit, pig, mouse, rat, guinea pig, hamster, dog, cat, etc.

In some cases, immune cells that are adversely affected or comprised will undergo apoptosis or cell death. This is common in many immune cells as a safety feature, since immune cells that have been damaged in some way may accidently become harmful to their host organism. In some cases, damaged or compromised immune cells may produce inflammatory cytokines to warn other immune cells. Examples of inflammatory cytokines include, but are not limited to, Tnf-alpha (Tnf-α), Cxcl1, Cxcl10, Type I interferons, interferon-betas, or the like. However, as the insertion of the microneedles into immune cells is largely a physical phenomenon, certain types of biological effectors may be inserted into the immune cells without causing increased inflammatory cytokine production or apoptosis, unlike in many prior art techniques.

Following the initial contact between the immune cells and the NW array, in some aspects of the invention, the immune cells are incubated for a period of time to allow for release of the molecules of interest into the penetrated immune cells from the NWs. For example, the cells may be incubated at a temperature of approximately 37° C., or other temperatures suitable for the cell type and organism from which the cell arises. The cells may be incubated for at least an hour, at least about 4 hours, at least about 12 hours, at least about a day, at least about 2 days, at least about 3 days, at least about 4 days, at least about 5 days, etc.

In some aspects of the invention, the phenotypes of the immune cells are then assessed. Examples of phenotypes include, but are not limited to, cell survival (e.g., whether the cell is alive or dead), ability of the cell to migrate, ability of the cell to divide, the production of one or more compounds (e.g., secreted by the cells), or the like. For example, the phenotype of an immune cell may be determined by analyzing immune cells for gene expression using a microarray. As used herein, “microarray” refers to a collection of DNA sequences attached to a solid surface. It should be appreciated that any method of using microarrays known to those of ordinary skill in the art may be used. The phenotype of a cell may also be assessed by other techniques known in the art, including, but not limited to, DNA sequencing, quantitative real-time polymerase chain reaction (qRT-PCR), NanoString nCounter Analysis, and the like.

Some aspects of the invention also relate to methods comprising assessment of whether an immune cell shows production of an inflammatory cytokine after insertion of a nanowire with or without molecular cargo. As used herein, a “cytokine” refers to a protein that is secreted by a cell of the immune system and that has an effect on other cells. Several non-limiting groups of cytokines include interleukins and interferons. Several non-limiting examples of interleukins include 1-18 (IL-1, IL-2, IL-3, IL-4, IL-5, IL-6, IL-7, IL-8, IL-9, IL-10, IL-11, IL-12, IL-13, IL-14, IL-15, IL-16, IL-17 and IL-18). IL-1 includes interleukin-1 alpha and interleukin-1 beta (IL-1 alpha and IL-1 beta). IL-5 is also known as eosinophil differentiation factor (EDF). IL-6 is also known as B-cell stimulatory factor-2 (BSF-2) and interferon beta-2. Several non-limiting examples of interferons include IFN-alpha (IFN-α), IFN-beta (IFN-β), IFN-omega (IFN-ω) and IFN-gamma (IFN-γ). Further examples of cytokines include TNF-alpha (TNF-α), TGF-beta-1 (TGF-β1), TGF-beta-2 (TGF-β2), TGF-beta-3 (TGF-β3) and vascular endothelial growth factor (VEGF).

In one set of embodiments, a substrate comprising nanowires, as discussed herein, may be used as a screening tool. For example, in some cases, a plurality of cell types may be determined or studied by applying a substrate comprising nanowires (e.g., having a biological effector) to the plurality of cell types. The response or phenotypes of the cells may be determined to determine the effect of the biological effector on the plurality of cell types, and in some cases, one or more cells or cell types may be selected based on such results. As another non-limiting example, a substrate comprising nanowires having coated thereon various biological effectors may be exposed to a population of cells to determine which of the biological effectors have a desired effect on the cells. For example, there may be 2, 3, 4, 5, 10, 20, 30, 50, 75, 100, or more biological effectors coated on various nanowires on the substrate.

The following documents are incorporated herein by reference in their entireties: U.S. patent application Ser. No. 13/264,587, filed Oct. 14, 2011, entitled “Molecular Delivery with Nanowires,” by Park, et al., published as U.S. Patent Application Publication No. 2012/0094382 on Apr. 19, 2012; International Patent Application No. PCT/US11/53640, filed Sep. 28, 2011, entitled “Nanowires for Electrophysiological Applications,” by Park, et al., published as WO 2012/050876 on Apr. 19, 2012; International Patent Application No. PCT/US2011/53646, filed Sep. 28, 2011, entitled “Molecular Delivery with Nanowires,” by Park, et al., published as WO 2012/050881 on Apr. 19, 2012; U.S. Provisional Patent Application Ser. No. 61/684,918, filed Aug. 20, 2012, entitled “Use of Nanowires for Delivering Biological Effectors into Immune Cells,” by Park, et al.; and U.S. Provisional Patent Application Ser. No. 61/692,017, filed Aug. 22, 2012, entitled “Fabrication of Nanowire Arrays,” by Park, et al. In addition, the following PCT applications, each filed on Mar. 15, 2013, are incorporated herein by reference in their entireties: “Fabrication of Nanowire Arrays,” by Park, et al.; and “Microwell Plates Containing Nanowires,” by Park, et al.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are incorporated by reference in their entirety.

### EXAMPLES

### Example 1

### Demonstration of the Capability of NWs to Deliver Biomolecules to a Wide Range of Immune Cells in an Effective, Minimally Invasive Manner

NWs were used to deliver biomolecules to several mature immune cell subsets, including bone marrow-derived dendritic cells (BMDCs, CD11c+), B cells (CD19+), dendritic cells (DCs, CD11c+), macrophages (Mphi (MΦ), CD11b+), natural killer cells (NK, DX5+), and T cells (CD4+), which were immunomagnetically isolated through magnetic activated cell sorting (MACS) from mouse bone marrow and spleen samples or from human blood samples. Following biomolecule delivery into the immune cells, the effectiveness of delivery and the effects on cell health, function, and viability were studied.

### Methods

### Primary Mouse Immune Cell Isolation and Culture

6-8 week old female C57BL/6J mice were obtained from Jackson Laboratories. BMDCs were generated. To isolate mouse primary immune cells, spleens were dissociated into single-cell suspensions by passage through a nylon mesh (BD Falcon). CD4+ T cells, B cells, NK cells, DCs, and macrophages (MΦ) were enriched via MACS separation with CD4, CD19, DX5, CD11c, and CD11b MicroBeads (Miltenyi Biotec) respectively. Prior to plating, sorted cell suspensions were filtered twice through 40 um nylon mesh to remove clumps. When extracting DCs, spleens were treated with 1 mg/mL Collagenase D in complete media at 37° C. for 20 minutes prior to dissociation to reduce clumping and debris. All splenic cells were cultured in BMDC without granulocyte macrophage colony-stimulating factor (GM-CSF).

### Primary Human B Cell Isolation and Culture

Heparinized blood samples were obtained from normal donors and patients enrolled on clinical research protocols at the Dana-Farber Harvard Cancer Center (DFHCC) approved by the DFHCC Human Subjects Protection Committee. Peripheral blood mononuclear cells (PBMC) were isolated by Ficoll/Hypaque density gradient centrifugation. Normal human B cells were immunomagnetically isolated with CD 19 MicroBeads (Miltenyi Biotec). All B cells were cultured in media with AIM-V media (Invitrogen) supplemented with 2 microgram/milliliter (μg/mL) IL-4 (R and D Systems), 5 microgram/milliliter (μg/mL) insulin (Invitrogen), and 50 microgram/milliliter (μg/mL) transferrin (Roche).

### Nanowire Fabrication & Functionalization

4×4 mm substrates displaying ordered arrays of Si NWs were fabricated by defining NW sites with photolithography, depositing an aluminum etch mask, reactive ion etching (RIE) to yield an array of three-dimensional NWs, and thermally oxidizing and thinning with RIE to obtain the desired NW sizes.

The physical parameters of the NWs were optimized for each of the immune cell types. In general, it was found that NW density, and to a lesser extent, diameter, needed to be scaled to match cell size, and that NW height required adjustment to facilitate cellular adhesion and penetration. For example, effective delivery of biomolecules into smaller immune cells that group in suspension, such as naive mouse B and T cells, required NWs that were longer (2-3 micrometers (μm)), sharper (diameter less than 150 nm), and denser (0.3-1 per micrometer² (μm²)), and also required an increased pre-incubation time to facilitate settling of those cells on top of the NWs. Larger, adherent immune cells (e.g., DC and mphi (MΦ)), on the other hand, required the use of NWs that were slightly shorter (1-2 micrometers (μm)) and less dense (0.15-0.2 per micrometer² (μm²)), and showed greater tolerance for slightly larger NWs (diameter less than 200 nm). While longer NWs (greater than 3 micrometers (μm)) proved minimally invasive to murine splenocytes and human B and T cells, they negatively impacted the viability of larger, adherent mouse and human immune cells (e.g., DC, mphi (MΦ), and BMDCs), possibly due to nuclear disruption. Substantial differences were also observed between mouse and human immune cells. Generally, human immune cells adhered more strongly with the NW substrate, with even small suspension human B and T cells anchoring well on short NWs (less than 1.5 micrometers (μm)). Conversely, murine B and T cells barely settled on the short NWs, even when given over an hour before media addition.

The NWs were silanized using 3-mercaptopropyltrimethoxysilane. Samples were then coated with 3 mL of an Alexa Fluor Maleimide (Invitrogen), prepared according to the manufacturer's recommendation. After 30 minutes, samples were washed thrice in distilled, sterile water, and blown dry.

Delivery of Biomolecules using Silicon NWs

Si NWs were precoated with various fluorescent molecules (5 microliters (μL) at 1 microgram/microliter (μg/μL)), including: plasmid DNA prelabeled with Label-IT Cy3 or CyS, siRNA labeled with Alexa Fluor 546 and Alexa Fluor 647, IgGs labeled with Alexa Fluor 488 and Qdot 585, Qdots 525 and 585, rhodamine labeled peptides, and recombinant fluorescent proteins. Cells were plated on top of the precoated NW samples.

siRNA

siRNAs were obtained from either Qiagen or Dharmacon. For all BMDC experiments, human B cell experiments and viability tests, 3 microliters (μL) of a 100 micromolar (μM) siRNA solution were used. For the murine ex vivo cell tests, three different concentrations of siRNA were tested: 100, 33, or 11 micromolar (μM). Of the cell types tested, only the B cells showed a concentration-dependent knockdown in the range tested. Transient transfection of C57BL/6 mouse embryonic fibroblasts (MEFs; from ATCC) was performed using either DharmaFECT 1 or 3 as per the manufacturer's instructions.

### Activation of Immune Cells

Unless otherwise specified, cells were stimulated for 4 h the day after being plated. The stimulation molecules and concentrations per sample were:

| Cell Type | Molecule |
|---|---|
| Mouse (Mm, Mus musculus) | 100 ng/mL LPS |
| BMDCs | |
| Mm B Cells | 100 ng/mL LPS and |
| | 25 ng/mL Mm IL-4 |
| Mm DC Cells | 100 ng/mL LPS |
| Mm Mphi (MΦ) Cells | 100 ng/mL LPS |
| Mm NK Cells | 20 ng/mL Mm IL-12 and |
| | 5 ng/mL Mm IL-18 |
| Mm T Cells | 10 mL Mm aCD3/CD28 |
| | Dynabeads and |
| | 1 ng/mL Mm IL-2 |
| Human (Hs, Homo sapiens) | 10 ng/mL IL-4 and |
| B Cells | 0.5 mg/mL Hs CD 40L |

Ultra-pure E. coli K12 LPS was obtained from Invitrogen; Hs CD 40L, Mm IL-4, Mm IL-12, and Mm IL-18 were obtained from R and D systems; Mm IL-2 and aCD3/CD28 DynaBeads were obtained from Invitrogen.

### Confocal Microscopy Analyses

The day after plating, cells were incubated in media containing 1:500 dilution of either: CellMask (Invitrogen), 1 Vybrant DiI (Invitrogen), 10 mg/mL Fluorescein Diacetate (FDA, Invitrogen), or 1 mg/mL Octadecyl Rhodamine B Chloride (r18) in absolute ethanol (Invitrogen). After a minute, samples were rinsed through PBS and then imaged using an upright confocal microscope (Olympus).

### Scanning Electron Microscope (SEM) Analyses

24 hours after plating, cells were fixed in a solution of 4% gluteraldehyde in 0.1 M sodium cacodylate for 2 hours, rinsed, and fixed again in a 1% solution of osmium tetroxide in 0.1 M sodium cacodylate for 2 hours. The samples were then dehydrated in gradually increasing concentrations of ethanol (from 50-100%) in water, dried in a critical point dryer, and sputter-coated with a few nanometers of platinum/palladium.

### Live-Dead Cell Imaging

The day after plating, the cells were cultured in 1 microgram/mL (μg/mL) Hoechst dye for 30 minutes. Immediately prior to imaging, each substrate was rinsed in PBS and placed in a live-dead staining solution of 2 micromolar (μM) EthD-1(Invitrogen) and either 2 micromolar (μM) Calcein-AM (Invitrogen) or 50 nM Fluorescein Diacetate (FDA) in PBS for one minute. After rinsing, each sample was imaged at a height of 5 micrometers (μm) above the substrate's surface using an upright confocal microscope equipped with a scanning stage (Olympus, Prior). To ensure that each sample was captured in its entirety, the imaging field was raster-scanned across each substrate using built-in multi-area viewing software (FV10, Olympus). At each location, three color (excitation wavelengths: 405 nm, 488 nm, and 559 nm) epifluorescence-like confocal images were captured by fully opening the system's pinhole. Each experimental condition and time point was repeated in triplicate. Values represent mean plus or minus (±) standard error of the mean.

The stack of images comprising each sample was analyzed using Matlab. For each sample, the live cell count was calculated by identifying the number of nuclei bound within a Calcein-AM or FDA positive cell that did not stain for EthD-1, while the total cell count was derived from the total number of nuclei, bound or unbound. To aid in counting adjacent cells with overlapping fluorescence profiles, histograms of nuclear size were fit with a constrained double Gaussian. Subsequently, nuclei were counted by binning using the fitted mean. Objects below half the mean were discarded as debris.

Importantly, independent samples were assayed at each time point; after 24 hours, samples that had been examined and returned to the incubator showed little to no live cells upon restaining and reimaging. This could be due to toxicity associated with either light exposure, the chemical stains themselves, or the duration of imaging (performed in room temperature PBS).

### CellTiter-Glo Viability Assay

In some instances, cell survival was measured using a luminescence cell viability assay that quantifies the amount of ATP present (CellTiter-Glo, Promega, Madison, Wis.) as per the manufacturer's recommendations, save minor modifications. In brief, samples on NWs were first moved from 48-well to 96-well plates which contained 100 microliters (μL) of prewarmed culture media in each well and were allowed to cool to room temperature. Then, 100 microliters (μL) of CellTiter-Glo reagent were added to each well, and the plate was mixed on an orbital shaker for 5 minutes. The total contents of the well (200 microliters (μL)) were then transferred to fresh, opaque 96-well luminescence measurement plates. Plates were read using a luminometer (Perkin Elmer TopCount, Perkin Elmer, Waltham, Mass.). An ATP standard curve was freshly generated for each experiment. All the conditions were run in triplicate and the data is represented as mean plus or minus (±) standard error of the mean.

qRT-PCR

NW substrates were removed from their original multiwell plates and, after being washed with PBS, placed into a 96-well plate. Subsequently, cells from each sample were lysed and their mRNA was extracted using a TurboCapture 96 mRNA kit (Qiagen). Next, cDNA was synthesized using a Sensiscript RT Kit (Qiagen). Quantitative RT-PCR was performed in either a 96 or a 384-well format. Knockdown was measured by comparing each value to the average obtained for six or more control samples. Error bars represent standard error.

For experiments testing the effects of NWs with and without molecular coating on cell activation, certain cell types did not show measurable cytokine mRNA levels prior to stimulation. In such instances, a Ct value of 40 was assigned.

### Nanostring Analysis

Expression levels for a 300 gene inflammatory and antiviral signature were examined in BMDCs plated on either glass, NWs, or NWs coated with non-targeting (NT) control siRNA, both in the presence and absence of a 4 hour LPS stimulation. The averages of two independent samples were plotted against one another using Matlab. 95% confidence intervals were computed by fitting a histogram built from the ratios of one sample's expression to its corresponding replicate over all conditions and genes.

### Results

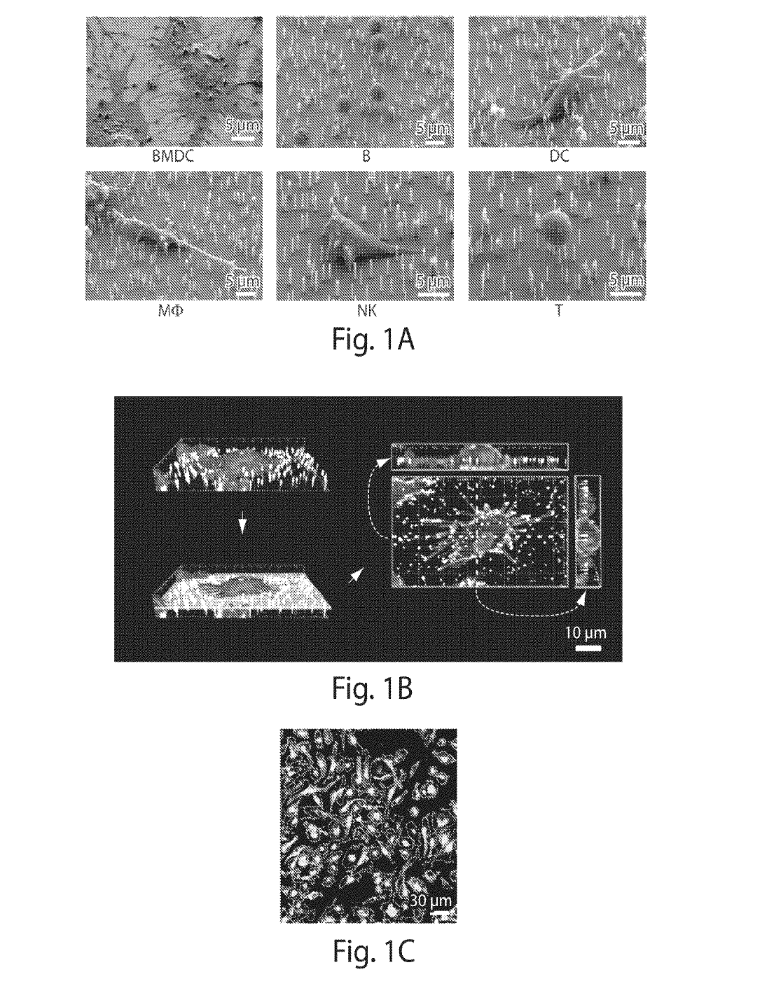

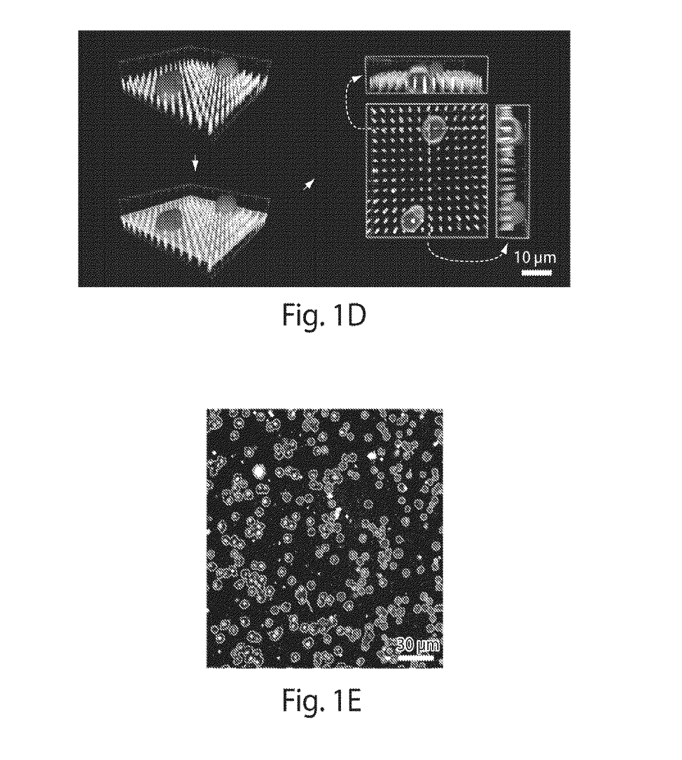

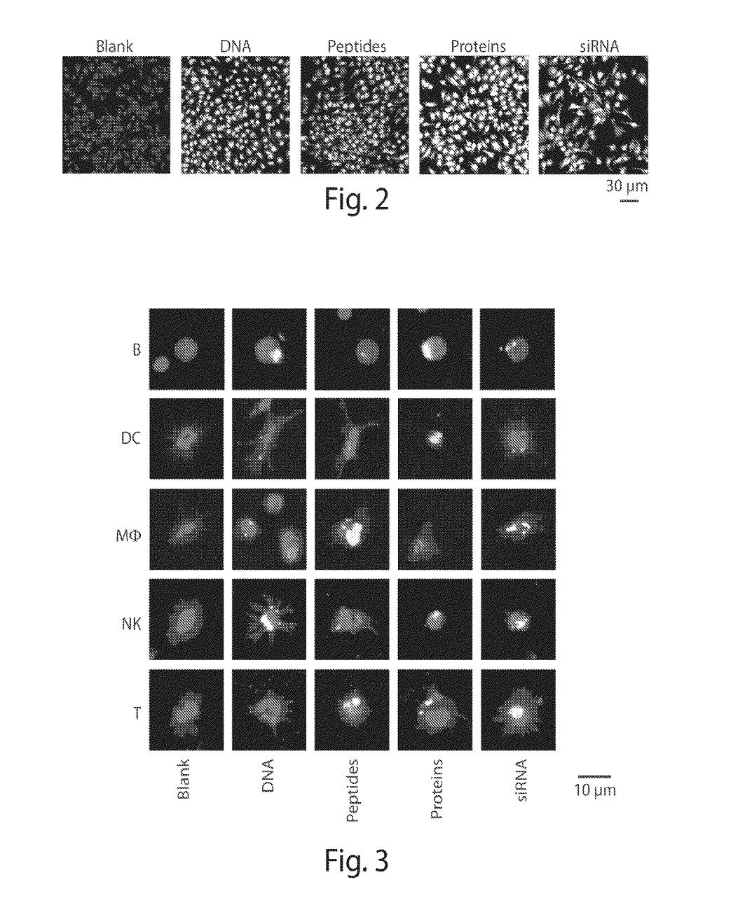

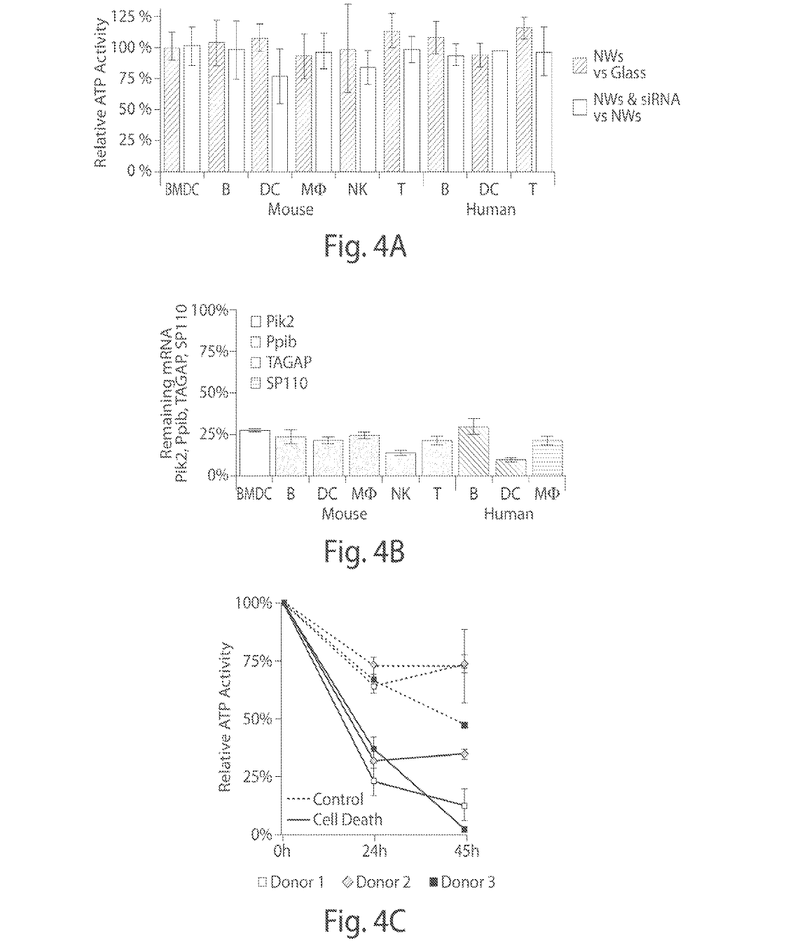

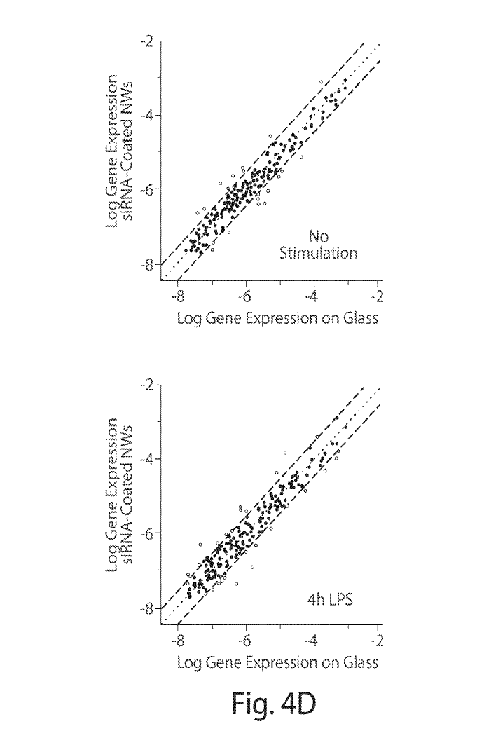

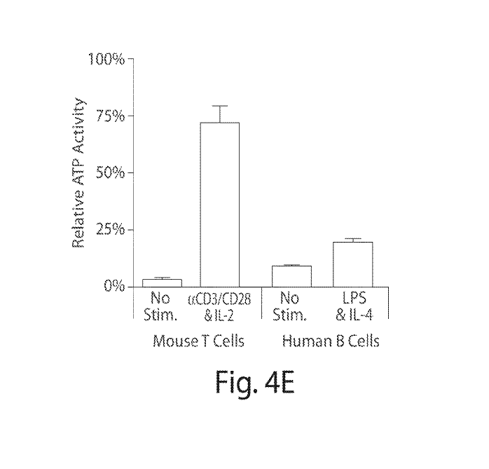

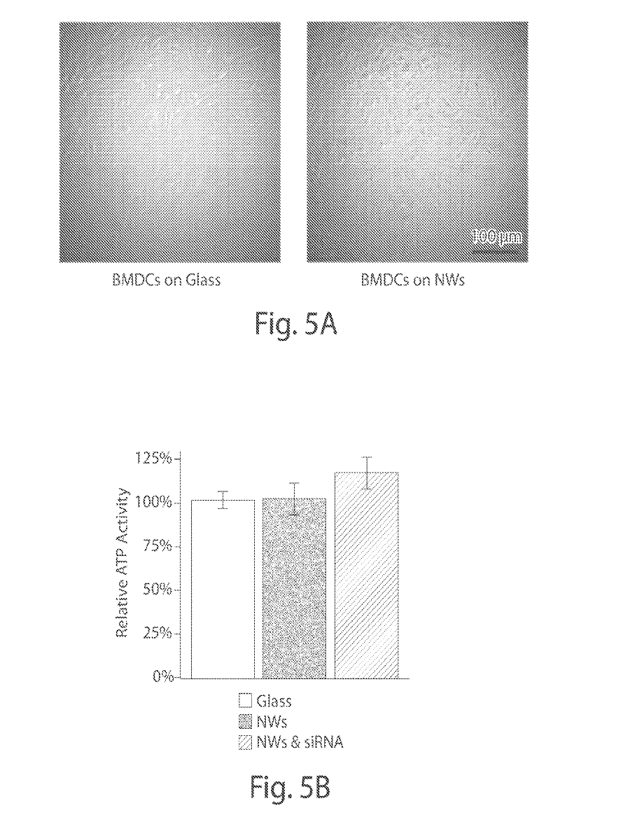

It was observed that NWs could consistently penetrate cellular membranes and deliver cargo without impacting cell health or morphology. FIG. 1a shows SEM images of BMDCs, B cells, DCs, mphis (MΦs), NK cells, and T cells on top of NW arrays 24 hours after plating, and FIGS. 1b and 1d respectively show three-dimensional reconstructions of confocally imaged mouse BMDCs and human B cells on top of NWs. When the NWs were pre-coated with fluorescently-labeled siRNAs, plasmids, peptides, and proteins, the molecules were delivered into nearly every cell without altering viability. FIGS. 1c and 1e show confocal microscope images showing delivery of siRNA to mouse BMDC and human B cells respectively, and FIGS. 2 and 3 show confocal scans showing delivery of DNA, peptides, proteins, and siRNA into BMDCs and primary murine splenocytes respectively. FIG. 4a shows that for a variety of mouse and human immune cells, plating cells on NWs does not diminish their viability (as measured by ATP activity) relative to glass controls (left), and coating the NWs with siRNA has negligible effect on cell health (right). In FIG. 5a, bright-field micrographs show little difference between BMDCs plated on glass (left) or NWs (right), and FIG. 5b shows little variation in ATP activity between BMDCs plated on glass (left), NWs (middle), or NWs coated with siRNA (right). It was also found that the biomolecular cargo delivered on the NWs was functional in the cells. In particular, siRNAs delivered on the NWs yielded substantial reductions (greater than 69%) in targeted mRNA levels followed by the expected phenotypic changes in every mouse and human immune cell type tested (FIG. 4b). And when cell viability was measured (as ATP activity) on 3 different sets of human B cells that received either non-targeting (NT) siRNA or cell death-inducing (CD) siRNA, it was found that the CD siRNA (solid lines) effectively killed more cells than the NT siRNA controls (dashed lines) (FIG. 4c).

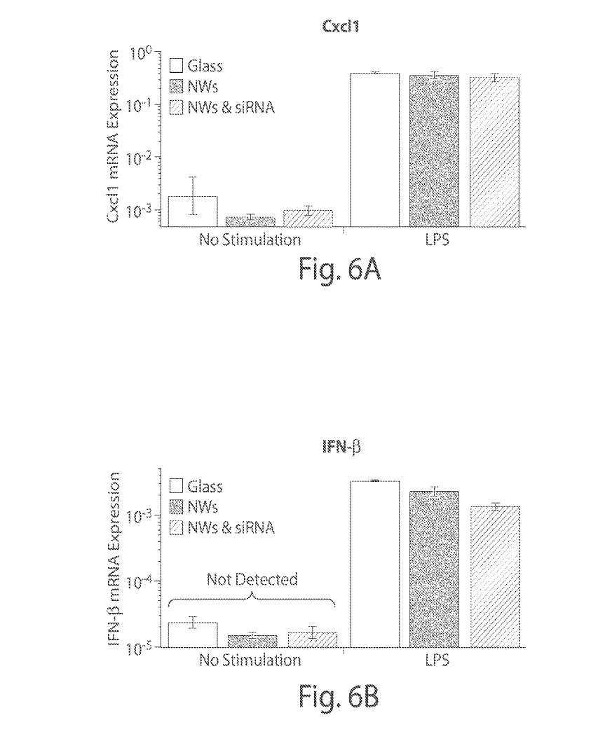

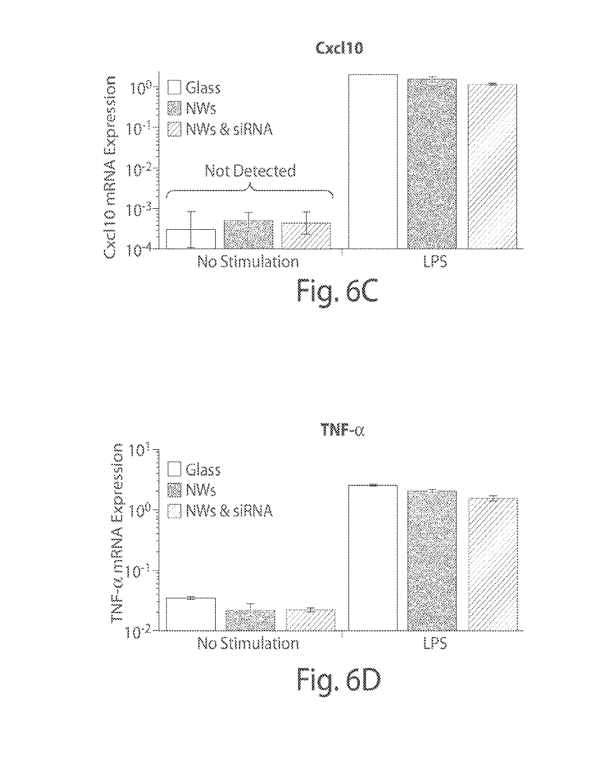

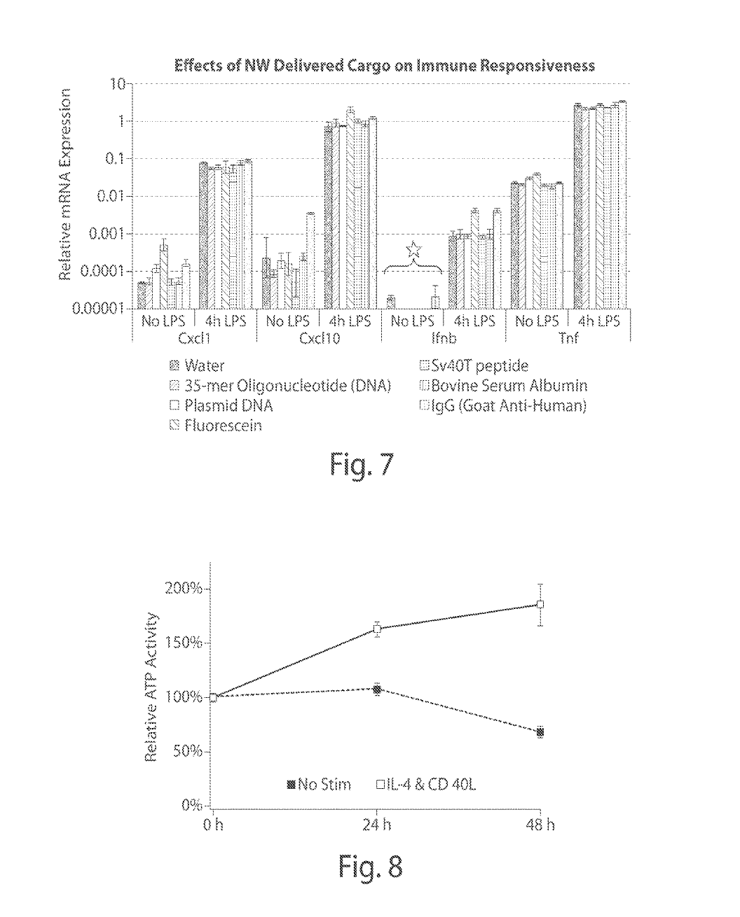

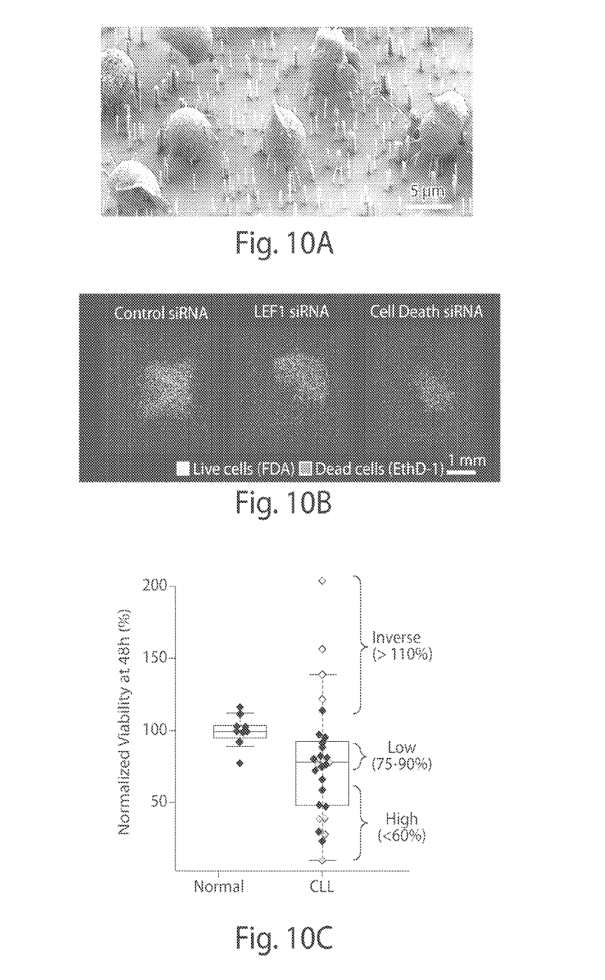

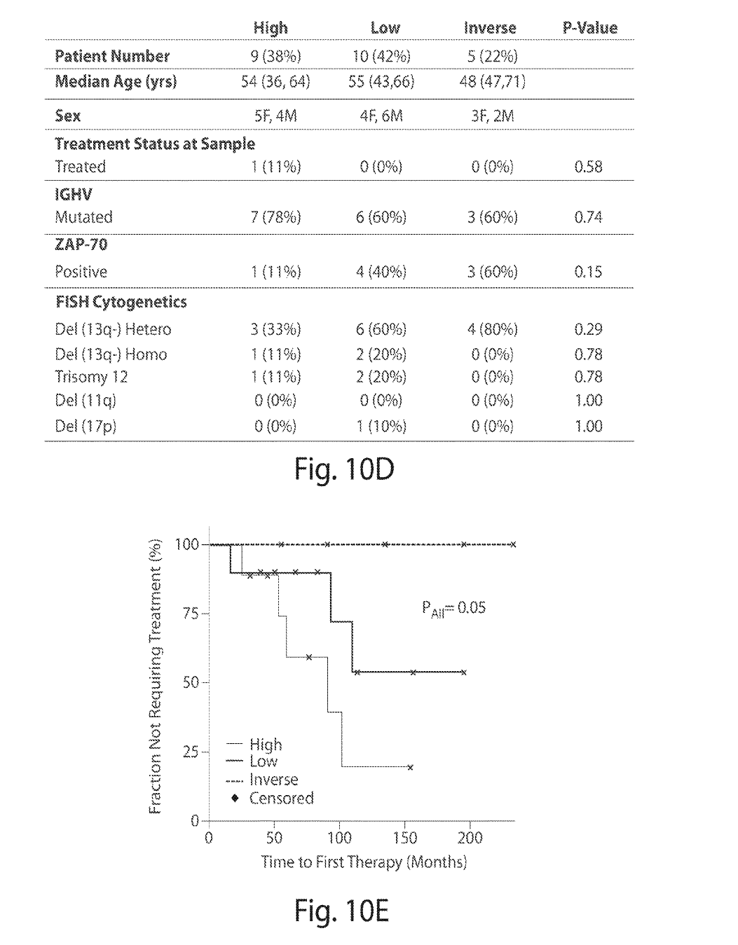

NW-mediated siRNA delivery neither activated an immune response in any of the tested cells nor interfered with normal immune sensing, cellular activation, or cell proliferation in response to physiological signals. First, when profiled with a signature set of 300 immune response genes (using the Nanostring nCounter technology), BMDCs plated on NWs coated with control siRNAs exhibited similar mRNA expression levels to BMDCs plated on glass, both pre-stimulation and in the presence of conventional stimuli, such as LPS (FIG. 4d). In FIG. 6, qRT-PCR results demonstrate that BMDCs, whether plated on glass (left), NWs (middle), or NWs coated with siRNA (right), did not show detectable levels of the major inflammatory cytokines Tnf-alpha (Tnf-α) and Cxcll or virally-induced Cxcl10 and Type I Interferons (Ifns; Ifn-beta (Ifn-β)) in the absence of stimulation, suggesting neither NWs nor their cargo strongly activate the endogenous antiviral or inflammatory pathways in cells. Similarly, FIG. 6 also shows that when stimulated with LPS, Cxcl1, Cxcl10, Ifn-beta (Ifn-β), and Tnf-alpha (Tnf-α) were robustly induced to equivalent levels for all samples, suggesting neither NWs nor their cargo inhibit immune response. FIG. 7 shows similar results for NWs delivering oligonucleotides, plasmid DNA, small molecules, peptides, and proteins. Without wishing to be bound by any theory, this may be due to the fact that NWs deliver cargo directly to the cytoplasm, and hence bypass the endosomal pathway, where innate immune sensing of double stranded RNA normally occurs. Finally, mouse T cells and human B cells were able to grow and divide on NWs in response to conventional stimulation (FIG. 4e). For example, FIG. 10, which shows relative ATP activity for human B cells that were not stimulated (bottom) or stimulated with IL-4 and CD40L (top), demonstrates that human B cells will grow and divide when stimulated.

The findings demonstrate that NWs provide a potent, yet minimally invasive, means of delivering perturbants into a variety of murine and human immune cells ex vivo. To date, although many methods—including electroporation/nucleofection, lipid vehicles, and viral transduction—have been tried for delivering molecular cargo to immune cells, none has been shown to be generally applicable across molecular species or immune cell types. In contrast, NW-mediated delivery worked for essentially all the cell types tested without affecting viability relative to multi-well or glass coverslip controls and did not activate innate immune responses. The ability to deliver functional biomolecular cargo in a minimally invasive fashion without activating immune cells or interfering with their ability to respond to physiological stimuli envisions the use of NW-based perturbations in studying the molecular circuitry governing immune cell activation and characterizing normal and diseased immune cells.

### Example 2

### Application of NW-Mediated Gene Silencing to Investigate the Role of the Wnt Signaling Pathway in Chronic Lymphocytic Leukemia (CLL)

NW-based delivery was used to investigate the potential basis of clinical heterogeneity in CLL. CLL, the most common adult leukemia in North America, is characterized by the progressive accumulation of dysfunctional mature B cells that have escaped normal apoptotic programs. Despite the fact that CLL-B cells of different patients share a common immunophenotype, CLL patients exhibit tremendous variability in their response to treatment and in their overall survival. While intensive research efforts over the past few decades have revealed much about this disease, a clear understanding of the intracellular circuitry responsible for CLL has yet to emerge. Analysis of microarray data from 193 CLL-B samples found overall dysregulation of the Wnt signaling pathway, which is normally responsible for guiding proliferation and cell fate, in CLL-B cells compared to normal CD19+ B cells. It was also found that LEF1, a terminal transcriptional activator of the Wnt signaling pathway previously linked to CLL-B cell survival, was one of the most upregulated mRNAs in CLL compared to normal B cells.

To determine the role of LEF1 in CLL-B cells, NW-mediated siRNA delivery was used to silence LEF1 expression in B cells isolated from 29 CLL patients and 12 normal donors, and cell survival was examined 48 hours after siRNA delivery.

### Methods

### Microarray Data Analysis of Wnt Dysregulation

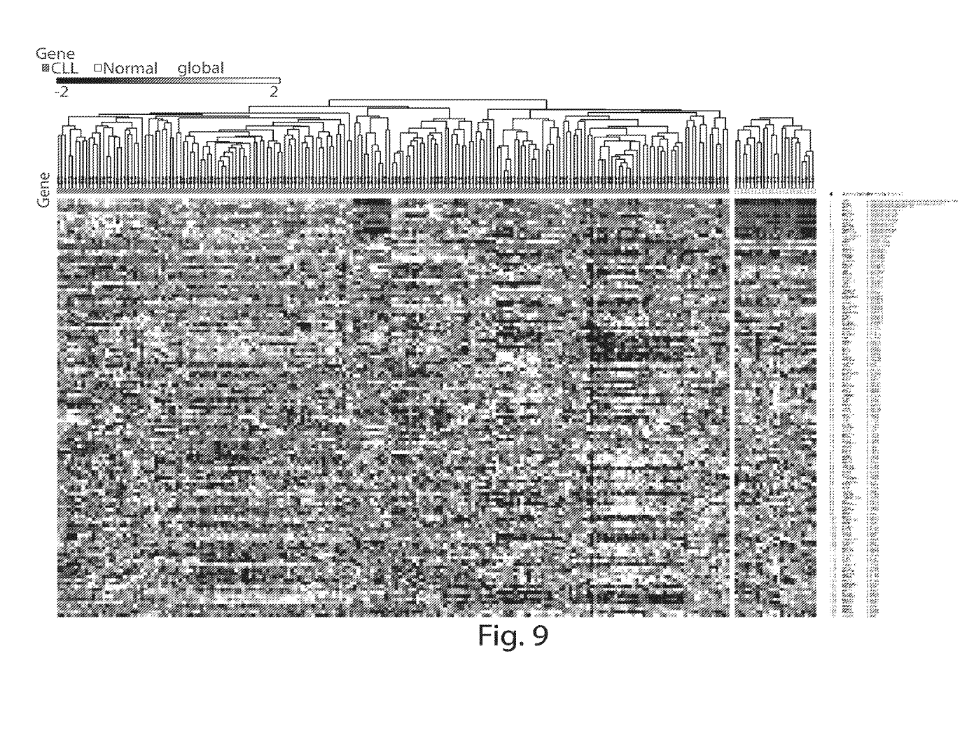

Total RNA was isolated from CLL cells (greater than 95% CD19+CD5+) using TRIzol reagent (Invitrogen), followed by column purification (RNeasy Mini Kit, Qiagen, Valencia Calif.). RNA samples were hybridized to Affymetrix U133A+ 2.0 arrays (Santa Cruz, CA) at the DFCI Microarray Core Facility. All expression profiles were processed using the robust multi-array average algorithm (RMA), implemented by the ExpressionFileCreator module in GenePattern, and Affymetrix probes were collapsed to unique genes (Gene Symbol) by selecting the probe with the maximal average expression for each gene. Batch effects were removed using ComBat, implemented by the ComBat module in GenePattern. Expression was globally (43%, 56 of 131 genes) dysregulated in the 193 CLL-B cell microarray samples relative to the 23 Normal donor controls (p less than 0.05; two-tailed Student's T-Test), with no discernible structure (FIG. 9). Among the Wnt pathway members, LEF1 was the most significantly dysregulated (p=1.78E-37).

NW-Mediated Delivery of LEF1 siRNA into Human B Cells

NW arrays were fabricated and functionalized using the methods described in Example 1, and the NWs were coated with LEF1 siRNAs. B cells were isolated from 29 CLL patients and 12 normal donors according to the methods described in Example 1, and the ex vivo human B cells were plated on top of the NW substrates, allowing the NWs to penetrate the cells and deliver the siRNA cargo. FIG. 10a shows an SEM image of CLL-B cells on top of NWs 24 hours after plating. The NWs successfully delivered functional siRNA into the B cells; for example, confocal images of CLL-B cells 24 hours after plating demonstrate that administration of a cell death inducing siRNA (far right) killed a larger number of cells than a non-targeting control siRNA (far left). The middle figure shows the effect of LEF1 siRNA on CLL-B cell viability for one particular patient sample.

### Microarray Data Analysis, Analysis of Variance (ANOVA), and Clinical Considerations

The 29 tested patient CLL-B samples were separated into three distinct classes based on the cells' survival in response to LEF1 silencing, and 4 samples were taken from each class for comparison of mRNA expression profiles. Genes significantly dysregulated between the three classes were identified using a one-way ANOVA. These 823 genes identified as significantly different between classes were analyzed using the Database for Annotation, Visualization and Integrated Discovery (DAVID) or Gene Set Enrichment Analysis (GSEA). This differentiating gene signature was subsequently used to classify 181 additional CLL patients as follows: (1) the Pearson correlation between each new patient and the 12 original samples was computed over the 823 ANOVA genes using z-scored expression data—z-scoring was performed to better weight each individual gene; (2) the average correlation for each new patient over the three groups was computed; and (3) new samples were assigned to the response class to which they showed highest average correlation. Samples were deemed unclassifiable if their average correlation value was lower than the highest average cross-correlation observed between any of the original 12 samples and the other two groups. Notably, reducing this requirement and assigning based upon highest average correlation alone still yielded a Kaplan Meier plot with three significantly different traces (p=0.0106, Logrank, FIG. 20), without any significantly enriched cytogenetic features. Finally, pursuing that sample analysis using a non-parametric ANOVA (Kruskal-Wallis) resulted in an 800-gene signature (547 gene overlap with the ANOVA list) and also yielded a significant Kaplan Meier curve with similar ability to classify additional CLL-B cell patients.

The three original groups of four microarray samples, as well as the larger correlated classes and the groups assigned based on knockdown, were compared for significant differences in the presence of known cytogenetic factors using 3×2 Fisher Exact tests in StataSE 10. Expression profiles were plotted using GENE-E or custom Matlab scripts. Data analysis, unless otherwise specified, was performed using Matlab.

A “single sample” extension of gene set enrichment analysis (SS-GSEA) implemented in R51 was used to test the intersection of either all annotated gene sets or those previously reported as stem cell gene sets and the ANOVA genes for differences in expression between the three response classes. Notably, the original 12 samples and extended classes showed enrichment for similar annotations, with the extended classes providing increase statistical power.

### Additional Statistical Considerations

Significances for the anti-survival effects of knocking down core Wnt pathway members in either CLL or Normal B cells relative to a non-targeting control siRNA were calculated using Wilcoxon signed rank tests. Normal and CLL B cells, meanwhile, were comparing using a Mann-Whitney rank sum test. Tests were performed using Stata SE or Matlab.

### Results

Using the methods described herein, it was found that CLL-B cells from different patients exhibited tremendous heterogeneity in their response to the knockdown of a single gene, LEF1. This functional heterogeneity defines three distinct patient groups not discernible by conventional CLL cytogenetic markers and provides a prognostic indicator for patients' time to first therapy. The findings highlight the opportunity for nanotechnology to drive biological inquiry in primary immune cells and tumors.

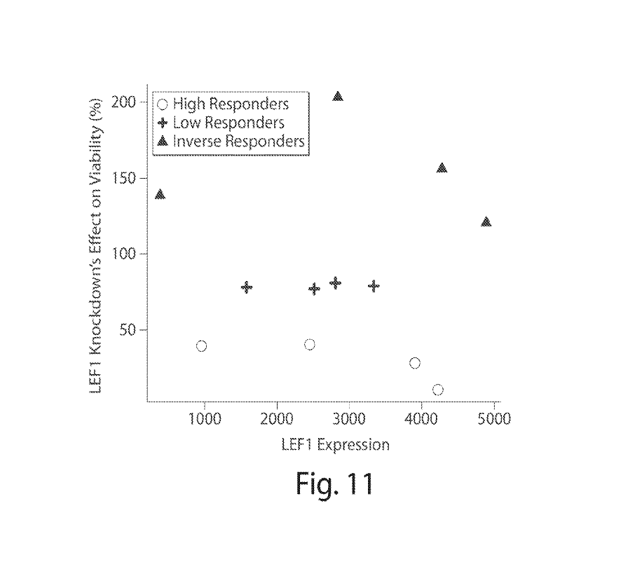

As a group, CLL-B cells exhibited lower viability (median 78%) upon LEF1 knockdown than CD19+ B cells from normal donors (100%) (p=0.004, Mann-Whitney rank sum test). This median response, however, did not fully capture the tremendous variation in the viability of different patients' CLL-B cells (ranging from 10 to 204%, FIG. 10c). Notably, the observed response heterogeneity did not correlate with patients' LEF1 expression levels, suggesting that the amount of LEF1 mRNA is not sufficient to explain the observed heterogeneity (FIG. 11).

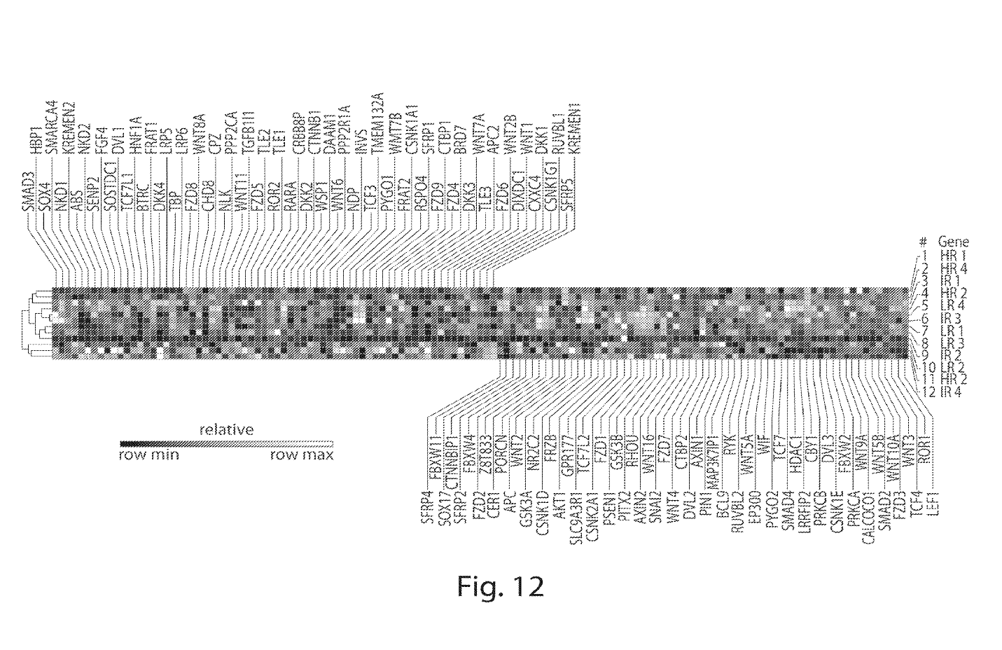

The 29 tested patient CLL-B samples were separated into three distinct groups based on the cells' survival in response to LEF1 silencing: high responders (HRs, n=9), whose CLL-B cell survival ratio (normalized to a non-targeting siRNA control) was less than 0.60; low responders (LRs, n=10), displaying a survival ratio between 0.75 to 0.90; and, inverse responders (IRs, n=5), with cell survival ratios in excess of 1.10 (FIG. 10c). Five samples with intermediate phenotypes were excluded from the analysis to generate more clearly defined classes. These three patient groups were not enriched for any known CLL-associated prognostic features, such as ZAP-70 or IgVH mutation status (FIG. 10d, Fisher's exact test, p greater than 0.05), and could not be predicted using simple unbiased correlation metrics, either genome-wide or based on Wnt pathway members (FIG. 12).

The patient groupings nevertheless exhibited statistically significant differences in their average time to first therapy (TTFT) (p=0.05, Logrank test); for HRs, TTFT was 67.5 months (4 of 9 right censored), while the TTFTs for LRs and IRs were 85.5 months (7 of 10 right censored) and 123.2 months (all 5 patients right censored), respectively (FIG. 10e). Strikingly, the results indicate that the response to even single-gene silencing can be used to predict the clinical course of CLL patients.

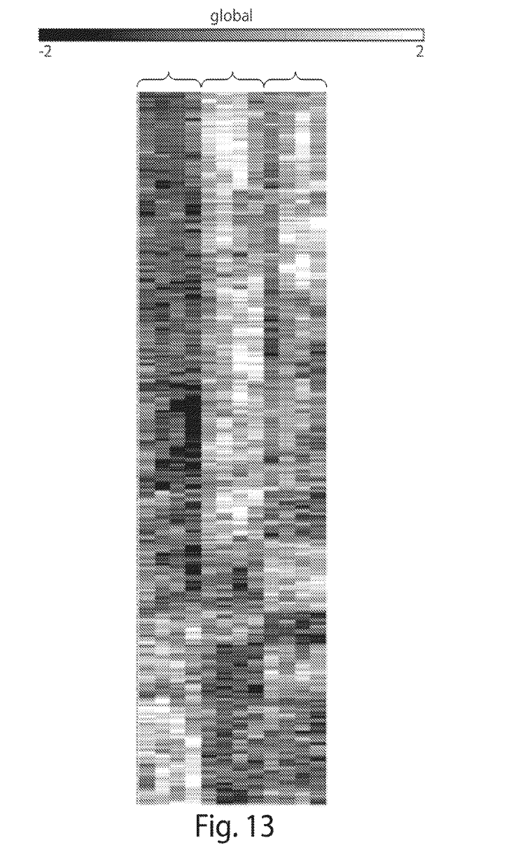

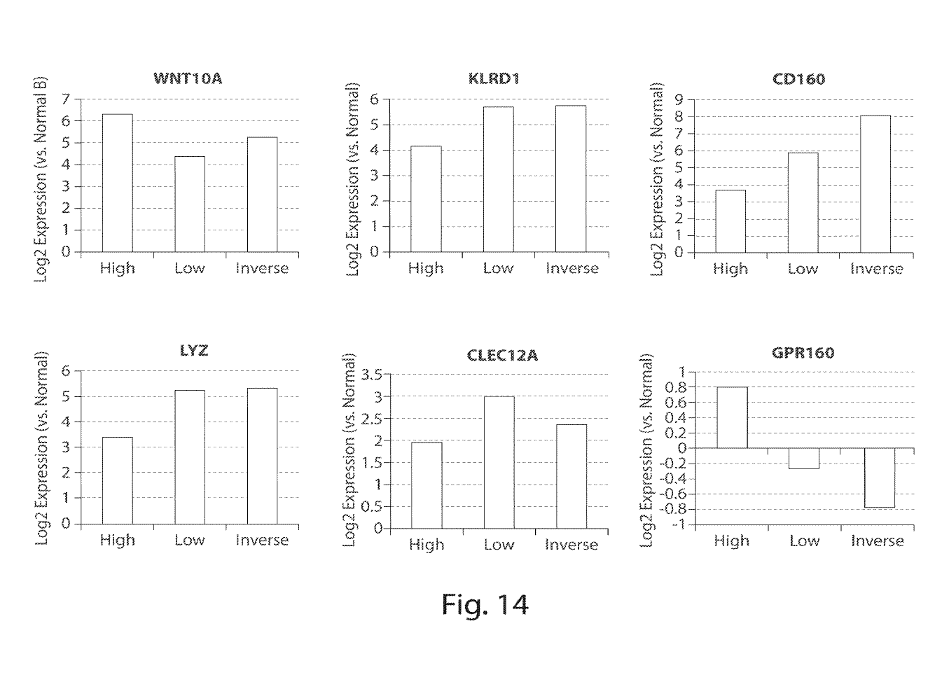

To examine the molecular basis of this surprising finding, the mRNA expression profiles from 12 of the 29 NW-tested samples (4 from each of the three classes for which microarray data were available) were compared using ANOVA. From this analysis, 823 genes (out of 20,766 total) were identified whose expression levels were significantly associated with the outcome of LEF1 silencing. From FIG. 13, which shows expression of the 823 genes for HRs (left), LRs (middle), and IRs (right), it can be seen that the expression signatures for HRs and LRs were dramatically different from one another; IRs were more similar to LRs, but displayed depressed expression across many more genes. The differences were validated by qRT-PCR for selected marker genes (FIG. 14).

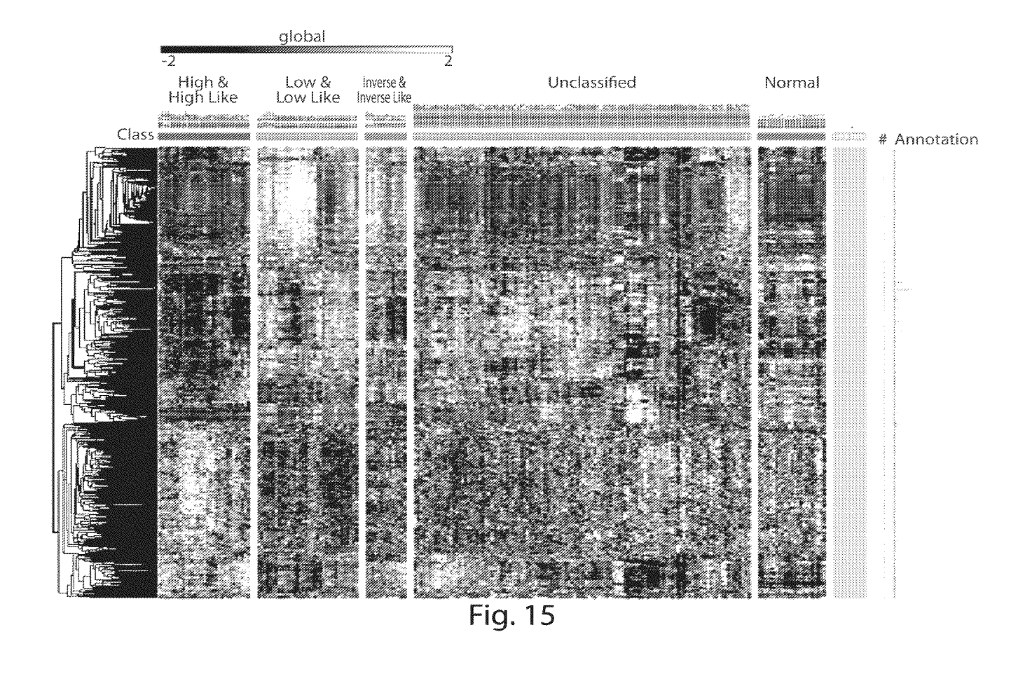

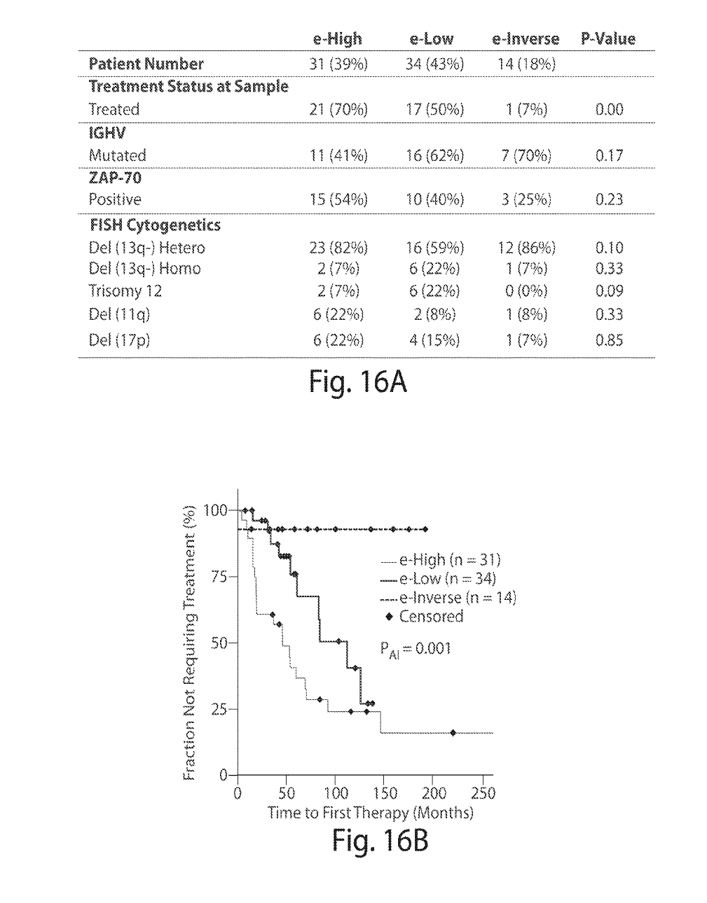

When the expression of the 823 genes was examined in an additional 181 CLL-B samples for which genome-wide expression profiles were available, 27 additional patients with gene expression patterns that resembled HRs (designated ‘high-like’) were found, while 30 and 10 additional patients showed patterns resembling LRs (‘low-like’) and IRs (‘inverse-like’) were found, respectively (FIG. 15). When the Kaplan-Meier analysis was performed on the extended patient groups (the original 12 patients from which the 823 gene set was identified plus the additional 67 patients identified among 181 patients), there were no observed enrichments for any known CLL-associated clinical prognostic markers (Fisher's exact test, p greater than 0.05, FIG. 16a), but there were significant differences in TTFT (p=0.001, Logrank test, FIG. 16b). These results suggest similarity between the extended groups and the tested samples.

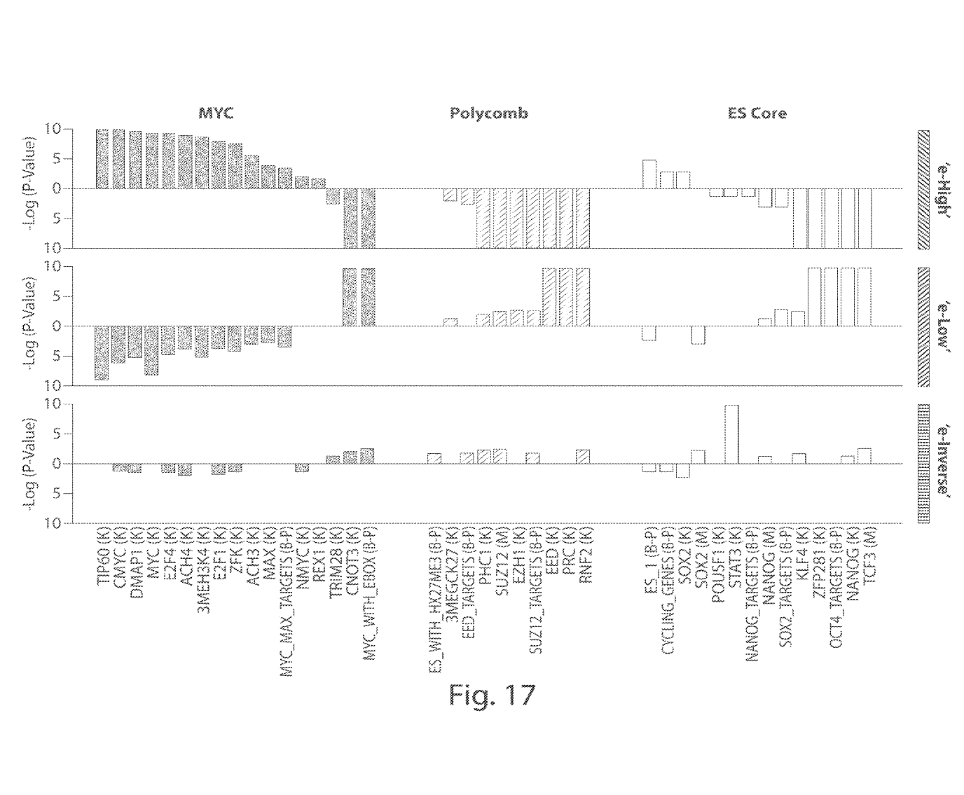

Several canonical pathways commonly linked to CLL and to malignancy were found to be enriched among the 823 genes using DAVID and single sample gene set enrichment analysis (SS-GSEA, FIG. 13). In particular, many of the 823 genes are associated with stem cell pathway regulation and hematopoietic lineage and development, consistent with the known roles of Wnt signaling. To explore this connection, SS-GSEA was used to compare expression levels of known gene sets that characterize hematopoietic (HSC) and embryonic stem (ES) cells—an ES core, a Polycomb repressor complex (PRC), and a MYC module—across the patient groups. In HRs and the high-like patient group, MYC and proliferation modules were elevated, whereas PRC and ES core modules were repressed, similar to previous observations in short-term HSCs and many aggressive cancers (FIG. 17). Conversely, LRs and the low-like group showed a signature that resembles self-renewing long-term HSCs, including increased PRC and ES core components and repressed MYC and proliferation genes. Finally, the IRs and the inverse-like group presented a less distinctive signature, save for the induction of genes targeted by STAT3.

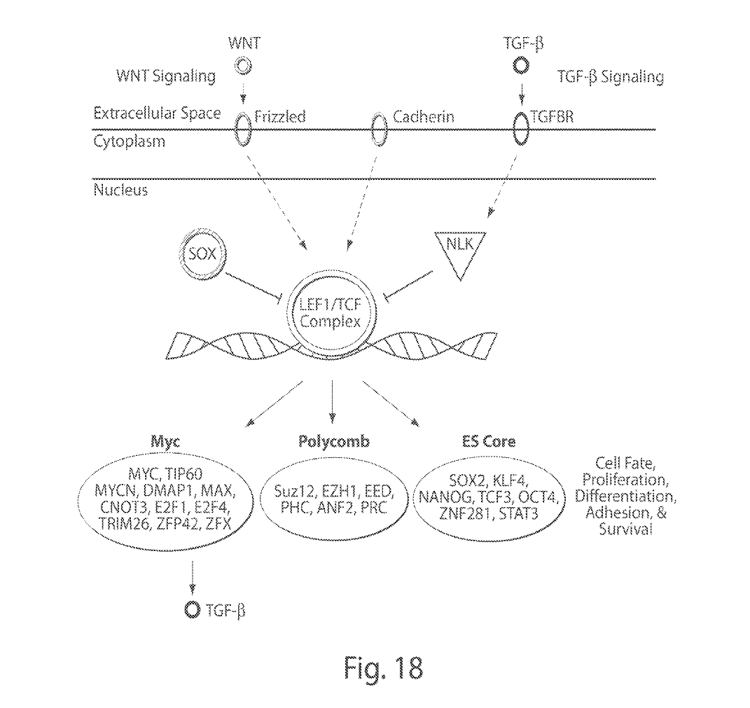

When integrated with information regarding the relative sensitivity toward LEF1 knockdown, the results of SS-GSEA suggest specific hypotheses on the pathways contributing to differentiating the three patient classes. Namely, the expression patterns and LEF1 sensitivity of HRs suggest that Wnt signaling may influence CLL pathogenesis via regulation of MYC by the LEF1/TCF complex. LRs and IRs, on the other hand, display enrichment for MYC targets with E-Box elements, such as TGF-beta1 (TGF-β1), suggesting an interplay between the Wnt and TGF-beta (TGF-β) signaling pathways. Elevated TGF-beta (TGF-β) signaling in LRs and IRs (FIG. 13) can, in part, explain the heterogeneity observed in response to LEF1 knockdown because the TGF-beta (TGF-β) pathway can influence the LEF1/TCF complex via negative feedback (FIG. 18).

Taken together, the results demonstrate that NWs provide a minimally invasive method for effectively delivering biomolecules into primary immune cells, including naive or resting cells, thereby enabling systematical analysis of cell circuits and functional responses in normal and malignant hematopoietic cells from both human and mouse. In particular, the studies demonstrate that response to NW-mediated gene silencing may be related to clinical parameters in CLL and can provide insight into the molecular circuitry contributing to disease heterogeneity. It is important to note that this NW-based perturbation strategy is fully extendable to other systems: starting from the cells taken from a single blood draw, NW-mediated gene silencing could be used to simultaneously probe the importance of each potential driver pathway of various hematological diseases, enabling not only the identification of gene signatures and pharmaceutical targets, but also the development of patient-specific combinatorial therapies.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method of identifying a therapeutic target for treating a disorder, comprising:
providing upstanding nanowires in an array;
coating the nanowires with a biological effector for modulating expression or activity of a cellular target;
contacting immune cells atop the array so that at least some of the immune cells are penetrated by one or more nanowires, the immune cells being related to the disorder;
incubating the immune cells for a period of time to allow for release of the biological effector into the penetrated immune cells;
assessing a phenotype of the immune cells; and
determining whether the cellular target is a therapeutic target for treating the disorder based on the phenotype,
wherein the average lengths, average diameters, and density of the nanowires are configured to permit adhesion and subsequent penetration of the immune cells.

2. The method of claim 1, wherein at least some of the nanowires are silicon nanowires.

3. The method of claim 1, wherein the biological effector is a small molecule, a DNA molecule, an RNA molecule, or a protein.

4. The method of any one of claim 1 or 2, wherein the average length of the nanowires is 0.1-10 micrometers (μm), and/or the average diameter of the nanowires is 50-300 nm, and/or the density of the nanowires is 0.05-5 nanowires per micrometer (μm²).

5. The method of claim 2, wherein the biological effector is a small molecule, a DNA molecule, an RNA molecule, or a protein.

6. The method of claim 3, wherein the average length of the nanowires is 0.1-10 micrometer (μm), and/or the average diameter of the nanowires is 50-300 nm, and/or the density of the nanowires is 0.05-5 nanowires per micrometer² (μm²).

7. The method of claim 5, wherein the average length of the nanowires is 0.1-10 micrometer (μm), and/or the average diameter of the nanowires is 50-300 nm, and/or the density of the nanowires is 0.05-5 nanowires per micrometer² (μm²)

8. A method of identifying a treatment for a disorder in a subject, comprising:
providing upstanding nanowires in an array;
coating the nanowires with a compound for treating the disorder;
contacting immune cells obtained from the subject atop the array so that at least some of the immune cells are penetrated by one or more nanowires, the immune cells being related to the disorder;
incubating the immune cells for a period of time to allow for release of the compound into the penetrated immune cells;
assessing a phenotype of the immune cells; and
determining whether the compound would be effective for treating the disorder in the subject based on the phenotype,
wherein the average lengths, average diameters, and density of the nanowires are configured to permit adhesion and subsequent penetration of the immune cells.

9. The method of claim 8, wherein at least some of the nanowires are silicon nanowires.

10. The method of claim 8, wherein the biological effector is a small molecule, a DNA molecule, an RNA molecule, or a protein.

11. The method of any one of claim 8 or 9, wherein the average length of the nanowires is 0.1-10 micrometer (μm), and/or the average diameter of the nanowires is 50-300 nm, and/or the density of the nanowires is 0.05-5 nanowires per micrometer² (μm²).

12. The method of claim 9, wherein the biological effector is a small molecule, a DNA molecule, an RNA molecule, or a protein.

13. The method of claim 10, wherein the average length of the nanowires is 0.1-10 micrometers (μm), and/or the average diameter of the nanowires is 50-300 nm, and/or the density of the nanowires is 0.05-5 nanowires per micrometer² (μm²).

14. The method of claim 12, wherein the average length of the nanowires is 0.1-10 micrometers (μm), and/or the average diameter of the nanowires is 50-300 nm, and/or the density of the nanowires is 0.05-5 nanowires per micrometer² (μm²).

15. A method of delivering a biological effector to immune cells, the method comprising the steps of:
providing upstanding nanowires in an array;
coating the nanowires with a biological effector;
contacting immune cells atop the array so that at least some of the immune cells are penetrated by one or more nanowires; and
incubating the cells for a period of time to allow for release of the biological effector into the penetrated cells,
wherein the average lengths, average diameters, and density of the nanowires are configured to permit adhesion and subsequent penetration of the immune cells.

16. The method of claim 15, wherein at least some of the nanowires are silicon nanowires.

17. The method of claim 15, wherein the biological effector is a small molecule, a DNA molecule, an RNA molecule, or a protein.

18. The method of any one of claim 15 or 16, wherein the average length of the nanowires is 0.1-10 micrometer (μm), and/or the average diameter of the nanowires is 50-300 nm, and/or the density of the nanowires is 0.05-5 nanowires per micrometer² (μm²).

19. The method of claim 16, wherein the biological effector is a small molecule, a DNA molecule, an RNA molecule, or a protein.

20. The method of claim 17, wherein the average length of the nanowires is 0.1-10 micrometer (μm), and/or the average diameter of the nanowires is 50-300 nm, and/or the density of the nanowires is 0.05-5 nanowires per micrometer² (μm²).

21. The method of claim 19, wherein the average length of the nanowires is 0.1-10 micrometers (μm), and/or the average diameter of the nanowires is 50-300 nm, and/or the density of the nanowires is 0.05-5 nanowires per micrometer² (μm²).

22. A method of silencing a gene in an immune cell, comprising:
providing upstanding nanowires in an array, at least some of the nanowires comprising siRNA coated thereon;
inserting at least one of the upstanding nanowires into an immune cell; and
incubating the immune cell for a time at least sufficient to activate the siRNA to silence the gene.

23. The method of claim 22, comprising:
inserting a plurality of the upstanding nanowires, each comprising the siRNA coated thereon, into a plurality of immune cells such that the gene is silenced by the siRNA in at least 90% of the immune cells.

24. The method of any one of claim 22 or 23, wherein the immune cell does not show production of an inflammatory cytokine after insertion of the nanowire.

25. The method of any one of claims 22-24, further comprising analyzing the immune cell for gene expression using a microarray.

26. The methods of any one of claims 22-25, further comprising analyzing the immune cell using qRT-PCR.

27. A method, comprising:
providing a plurality of substrates, each of which comprises upstanding nanowires in an array, at least some of which substrates comprise different biological effectors;
depositing a plurality of cells on the plurality of substrates to insert the biological effectors into the plurality of cells; and
determining phenotypes of the plurality of cells after insertion of the biological effectors.

28. The method of claim 27, wherein the biological effectors are inserted into at least about 90% of the cells.

29. The method of any one of claim 27 or 28, wherein substantially all of the plurality of cells are immune cells.

30. A method, comprising:
inserting a plurality of upstanding nanowires on a substrate into a plurality of immune cells, at least some of the nanowires being at least partially coated with a biological effector;
causing release of the biological effector internally of at least some of the immune cells; and
determining a phenotype of at least some of the immune cells.

31. The method of claim 30, comprising determining the phenotype using a microarray.

32. The method of any one of claim 30 or 31, wherein the biological effector comprises siRNA.

33. The method of any one of claims 30-32, wherein determining a phenotype comprises determining silencing of a gene within the immune cells caused by the biological effector.

34. The method of any one of claims 30-33, wherein at least some of the nanowires are silicon nanowires.

35. The method of any one of claims 30-34, wherein the biological effector is a small molecule, a DNA molecule, an RNA molecule, or a protein.

36. The method of any one of claims 30-35, wherein the average length of the nanowires is 0.1-10 micrometers (μm).

37. The method of any one of claims 30-36, wherein the average diameter of the nanowires is 50-300 nm.

38. The method of any one of claims 30-37, wherein the density of the nanowires is 0.05-5 nanowires per micrometer (μm²).
